(19) United States
(12) Patent Application Publication
(10) Pub. No.: US 2011/0136653 A1
(43) Pub. Date: Jun. 9, 2011

(54) METHOD FOR THE PRODUCTION OF A POROUS, CERAMIC SURFACE LAYER

(75) Inventors: Stefan Koebel, Dachsen (CH); Wolfram Weber, Hilzingen (DE); Wolfhart Rieger, Buch (CH)

(73) Assignee: Metoxit AG, Thayngen (CH)

(21) Appl. No.: 12/376,655

(22) PCT Filed: Aug. 8, 2007
(86) PCT No.: PCT/EP07/07023

§ 371 Date: Jun. 1, 2010

(30) Foreign Application Priority Data
Aug. 8, 2006 (DE) 10 2006 037 067.8

(51) Int. Cl. A61L 33/02; C04B 38/06

(52) U.S. Cl. 501/82; 427/2.24

(57) ABSTRACT

A method for producing a porous ceramic surface layer on a substrate includes providing a mixture including at least one polymer and at least one ceramic material and applying the mixture to the substrate.

### CROSS REFERENCE TO PRIOR APPLICATIONS

This application is a U.S. National Phase application under 35 U.S.C. §371 of International Application No. PCT/EP2007/007023, filed on Aug. 8, 2007 and which claims benefit to German Patent Application No. 10 2006 037 067.8, filed on Aug. 8, 2006. The International Application was published in German on Feb. 14, 2008 as WO 2008/017472 A2 under PCT Article 21(2).

### BACKGROUND

The present invention relates to a method for producing a surface layer. More specifically, present invention relates to a method for producing a porous ceramic surface layer for an implant.

### FIELD

Implants are becoming increasingly important in today's medicine. Implants come in a large variety of forms, such as medical implants (cardiac pacemakers, joint implants, dental implants), plastic implants (breast implants), functional implants containing RFID chips.

In general, implants are seen by the body as foreign objects. Because of this, the body may encapsulate an implant, so that, for example, no mechanically strong bond may be obtained between the bone and the implant.

In the prior art, the implant material is suitably selected such that the surface of the implant is colonized by tissue, e.g., bone cells. This is the case, for example, with titanium surfaces, and also with some oxide-ceramic implant surfaces. The mechanical strength of an interface produced is determined mainly by its topography. Therefore, dental implants made of titanium have structured surfaces.

Studies have shown that the pores of the surface must be of a certain size to achieve optimum mechanical strength for the bond between the implant and the bone.

In the prior art, pores in the surface of the implant are obtained by sandblasting and subsequent etching.

EP 1450722 A 1 describes a dental implant which is roughened to a surface roughness between 4 and 20 micrometers using a material-removal process.

DE 19858501 A2 describes a method for bioactivating ceramic implant surfaces by treatment with lye.

WO 2005/02771 A1 describes a two-stage process for applying a porous layer to an already porous surface.

However, the methods described in the prior art are of only very limited suitability for bioceramic materials. This is especially true for ceramic implants.

### SUMMARY

An aspect of the present invention to provide a method which will allow a porous ceramic surface layer to be produced on a substrate, in particular an implant, and which will further allow a predetermined implant surface porosity to be obtained by applying a single layer directly to the substrate.

In an embodiment, the present invention provides a method for producing a porous ceramic surface layer on a substrate. The method includes providing a mixture including at least one polymer and at least one ceramic material and applying the mixture to the substrate.

### BRIEF DESCRIPTION OF THE DRAWINGS

The present invention is described in greater detail below on the basis of embodiments and of the drawings in which:

FIG. 1 is a diagram showing the dispersion applied to a substrate;

FIG. 2 is a diagram illustrating the porous surface layer formed after the drying and sintering processes.

### DETAILED DESCRIPTION

In an embodiment, the present invention provides for a method for producing a porous ceramic surface layer (2) on a substrate (1), the method including the step of applying a mixture to the substrate (1), the mixture including:

at least one polymer and

at least one ceramic material.

A porous ceramic surface layer is understood to be a layer of a ceramic composition having a predetermined porosity and roughness to thereby enable re-growing tissue to interlock with an implant, creating a firm bond between the tissue or bone and the implant. The porous ceramic surface layer can be, for example, biocompatible, which means that re-growing tissue or bone can optimally grow into it, so that it is not rejected by the body.

In the present invention, the porous surface layer is obtained from a mixture, for example, after a drying process or after a sintering process.

A mixture is understood to be a material composed of at least two substances. Here, the mixture can be composed, for example, of at least one ceramic material and at least one polymer.

The mixture is applied, for example, to a substrate, and a predetermined surface roughness is formed subsequent to further processing steps, such as drying and/or sintering. The mixture further contains ceramic material; i.e., a ceramic.

A substrate is understood to be a material which has certain properties and to which the mixture is applied. The substrate has predetermined material properties (roughness, thermal expansion coefficient, chemical composition) to allow a porous ceramic surface layer to be suitably applied thereto, and to allow a bond to be created later between the substrate and said porous ceramic surface layer by sintering processes.

The substrate can include materials such as those used for implants, for example, a ceramic material; i.e., a technical ceramic from the group of oxide ceramics, such as Al₂O₃, ZrO₂, ZTA, ATZ, MgO, spinel, bioglass, nitride ceramics, such as Si₃N₄, and carbide ceramics, such as SiC, for example, Y-TZP type ZrO₂ having a density of at least 6.00 g/cm³. The substrate can have, for example, material properties (lattice constant, thermal expansion coefficient) similar to those of the porous ceramic surface layer.

The acronym ZTA stands for “zirconia toughened alumina”, which has a composition of, for example, 20% ZrO₂+80% Al₂O₃.

The acronym ATZ stands for “alumina toughened zirconia”, which has a composition of, for example, 80% ZrO₂+20% Al₂O₃.

A ceramic material is understood to include ceramic materials from the group which includes zirconia and which is used for medical and other technical applications. During sintering, the individual crystal groups, which initially are in powder form, bind together, resulting, for example, in the high strength of sintered ceramic materials. Sintered ceramic materials are also referred to as technical ceramics.

Application of the mixture to the substrate is understood to mean bringing the mixture into contact with the surface of the substrate to create a suitable bond between the two phases, i.e., the substrate and the mixture.

Application may, for example, be done directly. Direct application is understood to mean applying the mixture directly to the substrate, without any intermediate layer. Thus, the mixture is in direct contact with the substrate, so that the individual molecules or atoms of the substrate can interact directly with the molecules or atoms of the mixture, and similar material properties, such as lattice constants, may be used to advantage to provide an optimum bond of the later, porous ceramic surface layer to the substrate.

According to the present invention, the mixture can include, for example, at least one polymer and at least one ceramic material, which are mixed together such that the individual components are statistically nearly uniformly distributed per unit volume according to their mass proportions. An optimum mixing result may be obtained, for example, by heating the polymer to a predetermined temperature (for example, below 200° C.) and subsequently mixing it with the ceramic material in a suitable manner, which can be done using mixing methods known in the art.

Most polymers (except for fluorine-based polymers) undergo marked decomposition above 200° C. This temperature is dependent on the oxygen partial pressure. Therefore, under low-oxygen or oxygen-free conditions (such as vacuum), polymers become unstable only at higher temperatures.

A polymer is understood to be a chemical compound composed of chains or branched molecules containing the same or similar elements (monomers).

The polymer is selected, for example, from the group including polysaccharides, polyvinyl alcohols, wax emulsions, PMMA, cellulose fibers, polypropylene fibers, fatty alcohol sulfate preparations, or a combination thereof.

Polysaccharides, polyvinyl alcohols and wax emulsions are typical examples of temporary binding agents. In accordance with the present invention, pore-forming agents can be, for example, acrylic glass, cellulose fibers, polypropylene fibers and fatty alcohol sulfate preparations. As will be described later, the pore-forming agents in the present invention form the pores that will exist in the porous ceramic surface layer after the sintering process, because said pore-forming agents are vaporized or decomposed during the sintering process.

It is possible to add a single polymer or a plurality of different polymers to a particular mixture.

The advantage obtained by adding the polymer is that the polymer has certain material properties, such as the particle size of the polymer in the mixture, which, after the subsequent process steps, such as drying and sintering, significantly affect the porosity properties of the ceramic surface layer on the implant.

The polymer acts as a pore-forming agent, because during later sintering at a temperature of, for example, 1400° C., the polymer in the ceramic surface layer decomposes and vaporizes, allowing a porous ceramic surface layer that is free of elemental carbon to form on the substrate, in particular the implant.

The sintering temperature is dependent on the material itself and the particle size thereof. The finer the material, the lower the sintering temperature. It is also possible to lower this temperature by selectively adding sintering additives. For example, ZrO₂ and Al₂O₃, from a temperature range from 1200 to 1600° C., for example, from 1350 to 1450° C.

According to a further aspect of the present invention, the mixture can further contain, for example, at least one solvent.

H₂O can be used, for example, as the solvent. The purpose of adding the solvent to the mixture is to properly mix the polymer with the ceramic material and to partially dissolve it, the polymer remaining in a dispersive form, as has been explained earlier herein. The addition of the solvent (dispersant) to the mixture results in the formation of a dispersion. The advantage of this is that the mixture can be optimally applied in the form of a dispersion in one operation by a spraying or dipping process.

In the following, the term “dispersion” will be used in place of the term “mixture”, since a solvent was added to the mixture.

A dispersion is understood to be a two-phase or multiple-phase system in which a continuous phase (dispersant) contains additional phases (dispersing agents, particles).

In the dispersion, the polymer can, for example, be in the form of an emulsion or suspension and can be insoluble in the dispersant. Rather, the polymer can be in the form of spheres, so to speak, in the form of polymer spherules or particles of a certain size, the individual particles being spaced apart such that they do not form a continuous film. Thus, the ceramic material is in the interstitial spaces.

The polymer can, for example, be preheated prior to adding the solvent. However, the polymer may also be preheated and then mixed with the ceramic material and the solvent.

According to a further aspect of the present invention, the dispersion can, for example, contain at least one dispersing agent. A dispersing agent is understood to refer to additional phases in a dispersion. In the method of the present invention, it is preferred to use alkali-free polyelectrolytes, carboxylic acid preparations and alkanolamines. The dispersing agent serves primarily to make the dispersion more hydrophilic, thus allowing a larger amount of solid matter, such as a larger amount of ceramic material or inorganic binder, to be added to the dispersion, as will be described later in greater detail, providing for optimum mixing of the dispersion and improved dissolution of the individual components.

A deflocculant based on polyelectrolytes or carboxylic acid preparations can, for example, be used for purposes of deflocculation. Deflocculation is accomplished by electrolytic interactions. When the dissociated ions of the deflocculant contact the ceramic particles in the dispersion, the charge developing at the surface of the raw material particles in the aqueous system is equalized. The particles that are then present can pass each other more easily. The static charges of the raw material particles causing the particles to repel each other remain effective. This results in reduced viscosity. Non-oxides, such as silicon carbide and silicon nitride, are deflocculated and dispersed using alkanolamine-based raw materials. These deflocculants have a pseudo-cationic effect. Binding of the deflocculant to the anionic surface of the raw material to be deflocculated results in the formation of charges of equal sign, causing the raw material particles to repel each other. The distance between the raw material particles is increased, leading to a reduction in viscosity.

According to a further aspect of the present invention, the dispersion can, for example, also contain at least one inorganic binder. The inorganic binder is selected from the group of phosphates (e.g., Al-monophosphate) or silicates, or is a combination of these components. The inorganic binder allows the porous ceramic surface layer; i.e., the final product of to the present invention, to obtain high strength after completion of all processing stages; i.e., from the application of the dispersion to the substrate to the sintering process. Also, the inorganic binder causes the ceramic material; i.e., preferably Y-TZP type ZrO₂, to better adhere to itself during and after the drying process.

According to another aspect of the present invention, the ceramic material is, for example, selected from the group including ZrO₂ materials, such as Y-TZP, ATZ, Ce-TZP, PSZ, Ce-ATZ, Al₂O₃, spinel, hydroxylapatite, bioglass Si₃N₄, and other biocompatible ceramic materials.

Y-TZP stands for yttria stabilized tetragonal zirconia (TZP=tetragonal zirconia polycrystal). Unstabilized zirconia (ZrO₂) is monoclinic at room temperature and has a tetragonal structure at temperatures above 1170° C. Since the change of the crystal structure from the tetragonal phase to the monoclinic phase involves an increase in volume of about 3%, pure ZrO₂ is mixed with yttria to prevent later destruction during the sintering process. In the dispersion of the present invention, Y-TZP is used as a ceramic material having properties similar to those of the ceramic material of the implant. Once sintered, Y-TZP is a technical ceramic having chemical and physical properties which have proved to be very useful for surgical implant applications.

Moreover, the mixture or dispersion to be applied to the substrate and subsequently dried and sintered can, for example, be applied directly to the substrate; i.e., without any intermediate layer. The resulting advantage is that the material properties of the substrate underneath, such as lattice constants, thermal expansion coefficient, etc., can be optimally exploited. This results in improved adhesion of the porous ceramic surface layer to the substrate on the one hand and, on the other, in the porosity desired.

According to a further aspect of the present invention, the method can, for example, include a drying step subsequent to the step of applying the dispersion.

The purpose of the drying step is primarily to allow the dispersion applied directly to the substrate to properly dry after the dripping process, so as to form a suitable porous ceramic surface layer after a later sintering process. For drying, the substrate is introduced into a drying device known in the art, and is dried for a predetermined period of time at a constant or constantly increasing temperature, which reaches a maximum after a certain time.

According to yet another aspect of the present invention, the drying step can, for example, be carried out at a temperature between 20° C. and 70° C., such as between 40° C. and 60° C., or at 50° C. This advantageously allows the substrate, and the dispersion applied thereto, to be optimally dried according to the material properties.

In the method of the present invention, drying is performed to remove the dispersant from the layer. In this process, the temperature can, for example, be increased to thereby reduce the relative humidity of the surrounding air. If the temperature is increased above the vaporization point of the dispersant, the dispersant is vaporized. Moreover, the dispersant can also be removed from the layer using a partial vacuum, because a reduction in pressure causes a decrease of the vaporization temperature.

In the case that, for example, the ceramic component is introduced into a heated liquid polymer, the following system can be used: a ceramic component, e.g., Y-TZP, is introduced into a polymer along with a pore-forming agent, e.g., PMMA, which polymer is readily soluble in supercritical CO₂. This polymer acts as a dispersant for the ceramic material and the pore-forming agent, and can be washed out in a pressure chamber containing highly pressurized CO₂. Such as combination can also be used for water-soluble polymeric dispersants, which can be removed in a water bath analogously.

Such processes are known as “debindering” in ceramics engineering. However, the present invention uses an inorganic binder which cannot be removed in this way.

According to a further aspect of the present invention, the method can further include, for example, a sintering process. A sintering process is understood here to mean that the substrate, together with the dried dispersion applied thereto, is introduced into a suitable sintering device, such as is known in the art for similar sintering processes.

The sintering process can, for example, be carried out at a temperature between 1000° C. and 2000° C., such as between 1200° C. and 1600° C., or between 1350° C. and 1450° C. As explained earlier, the polymer particles, which are embedded in the now dried dispersion layer on the substrate, are decomposed or vaporized by the sintering process. The sintering temperature can, for example, be adapted to the particular vaporization temperature of the polymer; i.e., the sintering temperature can, for example, be above the decomposition or vaporization temperature of the polymer.

According to further aspect of the present invention, the polymer present in the dispersion can, for example, have a predetermined particle size, such as between 10 micrometers (μm) and 100 micrometers, or between 20 micrometers and 50 micrometers. The particle size of the polymer is understood herein to be the outer diameter of the polymer particle. A predetermined percentage of the polymers in the dispersion can, for example, have a certain particle size; i.e., that it have a certain distribution, such as a Gaussian distribution.

During sintering, the polymer spherules present in the solidified dispersion decompose or vaporize.

Above a certain temperature, the dispersion solidifies into a stable structure, the polymer spherules decompose or vaporize, and pores are formed in the ceramic layer, the pores being located both within the ceramic layer and at the surface thereof.

The pores formed near the surface reveal the original geometry of the polymer spherules. Through suitable selection of the geometry and size (10 to 100 micrometers) of the spherules, it is possible to selectively control the size of the pores formed in the surface layer during later sintering. Thus, the greater the diameter of the polymer spherules, the rougher will be the surface of the porous ceramic surface layer.

According to another aspect of the present invention, the application of the dispersion is accomplished by a dipping process, a spraying process, or a combination thereof.

A dipping process is understood to mean, for example, that the substrate is completely or partially immersed in a vessel containing the dispersion, so that the substrate can be completely or partially wetted by the dispersion.

A spraying process is understood to mean, for example, that the substrate is introduced into, or placed on, a suitable device allowing the dispersion to be uniformly sprayed on the substrate.

The dispersion may also be applied to the substrate using a combination of dipping and spraying processes.

If, in addition, a drying step is technically necessary because, for example, the layer does not dry fast enough under ambient conditions due to the composition of the mixture or dispersion, drying is preferably carried out after the coating step.

Thus, the suitable dipping and spraying processes allow the dispersion to be applied in an efficient, economical and time-saving manner.

According to a further aspect of the present invention, additional ceramic components can, for example, be added to the dispersion. These ceramic components can, for example, be selected from the group which includes oxides, hydroxides, phosphates, and carbonates and which, in addition, enables functionalization of the porous layer. It is possible to add to the dispersion an additional, ceramic material which reacts chemically with the inorganic binder and/or other components of the dispersion.

According to a yet another aspect of the present invention, salts which are soluble in the dispersant, for example, be additionally added to the dispersion. During drying and subsequent sintering, these salts form substances which promote the biocompatibility and the healing-in of the coating, or the general properties thereof (strength, adhesion to the substrate, etc.).

According to a further aspect of the present invention, the porous ceramic surface layer can, for example, be applied to the substrate after a hot isostatic compaction (HIP) step and before an re-oxidation firing step.

According to another aspect of the present invention, the porous ceramic surface layer and the substrate together form, for example, an implant, such as a dental implant. Due to the material properties of both the substrate and the porous ceramic surface layer, an implant is obtained which has a biocompatible surface layer that allows re-growing tissue or bone to grow into, or bond to, the implant, resulting in optimum mechanical strength between the implant and the tissue or bone.

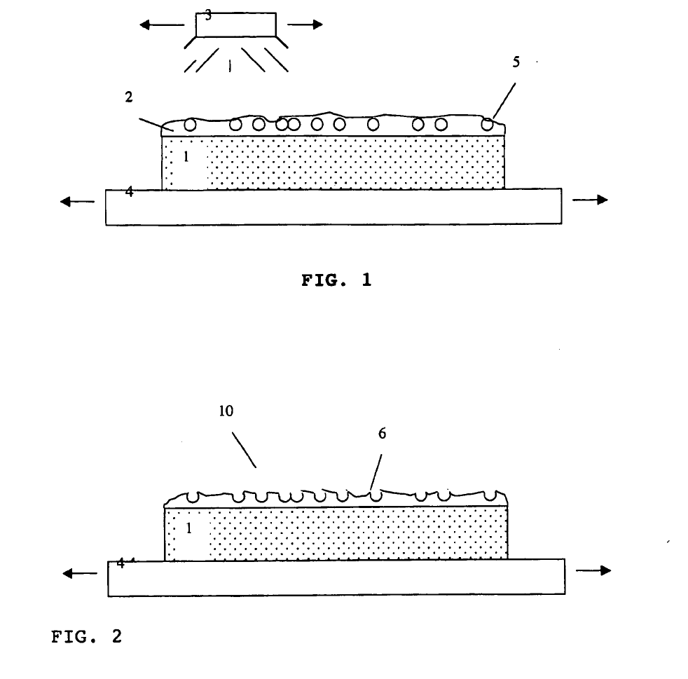

The diagram of FIG. 1 shows a dispersion 2 according to the present invention applied to a substrate 1, the application of dispersion 2 being accomplished using a spraying device 3.

In a first step, a dispersion preferred according to the present invention is made using the following components:

300 g of Tosoh TZ-3YS-E, as the Y-TZP type ceramic material, and 2.5 g of CaCO₃, 90 g of BAC-60, which forms the polymer spherules as the later pore-forming agent after the sintering process, 15 g of Butapur-1 as the inorganic binder, 3 g of Dispex® A40 as the dispersant, and 529 g of H₂O as the solvent, are introduced into a vessel and mixed until a suitable dispersion of uniform distribution is obtained. A uniform distribution is understood to mean that the individual components in the dispersion are present in statistically nearly equal proportions per unit volume

Subsequently, the dispersion is filled into a vessel (not shown) of a spraying device 3, which also has a mixing device (not shown) to maintain the dispersion in a well-mixed condition.

Substrate 1 is placed on a holder 4, the substrate being a Y-TZP type ceramic substrate called Metoxit TZP-A and having a density of at least 6.00 g/cm³.

The arrows in the diagram indicate that, depending on the particular application, both holder 4 and spraying device 3 can be moved relative to each other to achieve optimum application of dispersion 2 to substrate 1. In this exemplary embodiment, spraying device 3 was moved relative to holder 4.

After 5 ml of the dispersion were applied directly to substrate 1 (i.e., without any intermediate layer), a liquid surface layer of dispersion 2 formed on substrate 1. The liquid layer of the dispersion on the substrate has a thickness of 80 to 100 micrometers.

FIG. 1 also shows the polymer spherules 5 present in dispersion 2. The polymer spherules are statistically distributed in the dispersion layer on substrate 1, both within the layer and near the surface thereof.

Not shown are the other components contained in the dispersion, such as ceramic material, inorganic binder, or dispersing agent.

FIG. 2 shows the porous surface layer after a drying process and a sintering process.

Initially, a dispersion is prepared having the following composition:

300 g of Tosoh TZ-3YS-E, as the Y-TZP type ceramic material, and 220 g of BAC-60 as the pore-forming agent, 15 g of Butapur-1 as the inorganic binder, 3 g of Dispex® A40 as the dispersant, and 1200 g of H₂O as the solvent, are introduced into a vessel and mixed until a suitable dispersion of uniform distribution is obtained. The preferred composition of the dispersion suitably allows the dispersion to be applied using a dipping process, this dispersion being less viscous than that described in FIG. 1.

Subsequently, the front surface of a Y-TZP type substrate called Metoxit TZP-A and having a density of at least 6.00 g/cm³ is immersed (not shown) in the vessel containing the dispersion, so that the front surface of the substrate is covered with a 80 to 100 micrometer thick layer of the dispersion.

After that, substrate 1, together with the dispersion applied thereto, is dried in a drying chamber for 30 to 60 minutes at 50° C. until the dispersion has suitably solidified by solvent evaporating from the dispersion (not shown). This is done using a drying chamber such as is commonly used in the art.

After the drying process, substrate 1 carrying the solidified dispersion is introduced into a sintering device such as is commonly used in the art.

The sintering device is heated to a temperature of 1400° C. In the process, the substrate carrying the solidified dispersion is treated for a period of about 30 minutes, during which the components present in the dispersion are solidified into a porous ceramic structure, providing a porous ceramic surface layer.

Polymer spherules which have a particle size of 50 micrometers and are still present in the solidified dispersion have already decomposed at this temperature, because the decomposition of this polymer occurs at a much lower temperature (about 200° C.).

Because the dispersion solidifies into a certain stable structure and the polymer spherules decompose above a certain temperature, pores 6 are formed in the ceramic layer, which pores are located both within the ceramic layer and at the surface thereof. As a result, superficial and near-surface pores are produced which reveal the original geometry of the polymer spherules and which are formed by the sintering process alone, because the polymer spherules have decomposed long before the temperature reaches 1400° C. Thus, by selecting the geometry and size (10 to 100 micrometers) of the spherules, it is possible to selectively control the size of the pores formed in the surface layer during later sintering. Thus, the greater the diameter of the polymer spherules, the rougher will be the surface of the porous ceramic surface layer.

FIG. 2 further shows that the porous ceramic surface layer has a smaller thickness than liquid dispersion 2 because of the drying and sintering processes, during which the layer thickness decreases to about 20 micrometers.

Following are two tables for preferred exemplary embodiments of dispersion solutions.

Table A shows the compositions of three exemplary embodiments intended for application by spraying, while Table B shows the compositions of three exemplary embodiments intended for application by dipping.

TABLE A
Spraying process
(all FIGS. are given in grams)

| | EXAMPLE | | |
|---|---|---|---|
| | 1 | 2 | 3 |
| TZ-3YS-E | 300 | 300 | 300 |
| CaCO₃ | 2.5 | 5 | 15 |
| BAC-60 | 90 | 90 | 90 |
| (pore-forming agent) | | | |
| Butapur-1 | 15 | 15 | 15 |
| (inorganic binder) | | | |
| Dispex ® A40 | 3 | 3 | 3 |
| H₂O | 529 | 529 | 529 |

TABLE B
Dipping process
(all FIGS. are given in grams)

| | EXAMPLE | | |
|---|---|---|---|
| | 1 | 2 | 3 |
| TZ-3YS-E | 300 | 300 | 300 |
| CaCO₃ | | | |
| BAC-60 | 86 | 132 | 220 |
| (pore-forming agent) | | | |
| Butapur-1 | 15 | 15 | 15 |
| (inorganic binder) | | | |
| Dispex ® A40 | 3 | 3 | 3 |
| H₂O | 1200 | 1200 | 1200 |

The present invention is not limited to embodiments described herein; reference should be had to the appended claims

### LIST OF REFERENCE NUMERALS

1 substrate
2 dispersion
3 spraying device
4 holder
5 polymer spherules
6 pore
7 porous surface layer

1-17. (canceled)

18. A method for producing a porous ceramic surface layer on a substrate, the method comprising:
providing a mixture including at least one polymer and at least one ceramic material; and
applying the mixture to the substrate.

19. The method recited in claim 18, wherein the mixture further includes at least one solvent.

20. The method recited in claim 18, wherein the mixture further includes at least one dispersant.

21. The method recited in claim 18, wherein the mixture further includes at least one inorganic binder.

22. The method recited in claim 18, further comprising drying after the mixture is applied to the substrate.

23. The method recited in claim 22, wherein the drying is carried out at a temperature of between 20° C. and 140° C.

24. The method recited in claim 22, wherein the drying is carried out at a temperature of between 40° C. and 120° C.

25. The method recited in claim 18, further comprising sintering.

26. The method recited in claim 25, wherein the sintering is carried out at a temperature of between 1000° C. and 2000° C.

27. The method recited in claim 18, wherein the substrate includes at least one of an oxide ceramic, Al₂O₃, ZrO₂, ZTA, ATZ, MgO, spinel, bioglass, a nitride ceramic, Si₃N₄, a carbide ceramic, SiC, a biocompatible ceramic material and a non-biocompatible ceramic material.

28. The method recited in claim 20, wherein the dispersant includes a second polymer.

29. The method recited in claim 28, wherein the second polymer has a predetermined particle size.

30. The method recited in claim 29, wherein the particle has a size in a range of from 10 μm to 100 μm.

31. The method recited in claim 28, wherein the second polymer includes at least one of polyvinyl acetate, cellulose polysaccharide, polyvinyl alcohol, a wax emulsion, acrylic glass, a cellulose fiber, a polypropylene fiber, and a fatty alcohol sulfate preparation.

32. The method recited in claim 28, wherein the second polymer is different from the at least one polymer.

33. The method recited in claim 18, wherein the ceramic material includes at least one of a ZrO₂ material, Y-TZP, ATZ, Ce-TZP, PSZ, Ce-ATZ, Al₂O₃, spinel, hydroxylapatite, bioglass, Si₃N₄, and a biocompatible ceramic material.

34. The method recited in claim 21, wherein the inorganic binder includes at least one of a phosphate, silicate, carbonate and a sulfate.

35. The method recited in claim 19, wherein the solvent includes H₂O and an organic solvent having a hydrophilic functional group.

36. The method recited in claim 18, wherein the applying the mixture to the substrate is accomplished by at least one of dipping and spraying.

37. An implant comprising a substrate and a porous ceramic surface layer wherein the porous ceramic surface layer includes at least one polymer and at least one ceramic material.
